SEQUENCE LISTING (2 sequences)

SEQ ID NO: 1 - DNA, 36 bp, Artificial Sequence
  primer
aagcttatggaaatctgcagaggcctccgcagtcac

SEQ ID NO: 2 - DNA, 36 bp, Artificial Sequence
  primer
gtcgacctactcgtcctcctggaagtagaatttggt

(19) United States
(12) Patent Application Publication
(10) Pub. No.: US 2003/0130223 A1
(43) Pub. Date: Jul. 10, 2003

(54) Gene therapy composition for treating arthritis

(76) Inventors: Jong-Mook Kim, Seoul (KR); Seong-Hyun Ho, Incheon (KR); Woong Hahn, Seoul (KR); Sunyoung Kim, Seoul (KR)

Correspondence Address: David A. Einhorn, Esq., Anderson Kill & Olick, P.C., 1251 Avenue of the Americas, New York, NY, 10020, US

(21) Appl. No.: 10/331,446

(22) Filed: Dec. 27, 2002

Related U.S. Application Data
(60) Provisional application No. 60/344,316, filed on Dec. 28, 2001.

(51) Int. Cl. A61K 48/00

(52) U.S. Cl. 514/44

(57) ABSTRACT

An intramuscular injection composition for treating arthritis in a mammal, which comprises a DNA encoding interleukin-1 receptor antagonist (IL-1Ra) and pharmaceutically acceptable carriers

### CROSS REFERENCE TO RELATTED APPLICATION

This application is a non-provisional application of U.S. Serial No. 60/344,316, which was filed on Dec. 28, 2001.

### FIELD OF THE INVENTION

The present invention relates to an intramuscular injection composition for treating arthritis which comprises a DNA encoding interleukin-1 receptor antagonist; and a method for treating arthritis by injecting same into the muscles.

### BACKGROUND OF THE INVENTION

Rheumatoid arthritis (RA) is a chronic disease characterized by inflammation of the joints with concomitant destruction of cartilage and bone (Kaklamanis P. M., Clin. Rheumatol, 11: 41-7(1992)). Although the causes of RA are not fully understood, various experimental and clinical studies have suggested that proinflammatory cytokines, particularly interleukin-1 (IL-1) has an important role in RA pathogenesis(Arend W P and Dayer J M, Arthritis Rheum, 38: 151-60(1995); Arner E C, Arthritis Rheum, 32: 288-97(1989); Miyasaka N, et al., Arthritis Rheum., 31: 480-6(1988); and van de Loo F A, et al., J Rheumatol., 19: 348-56(1992)). It is well known that IL-1 can stimulate monocytes, recruit inflammatory cells, and induce secretion of factors that degrade cartilage (Dinarello C A, Blood, 77: 1627-52(1991)). In animal studies, systemic administration of IL-1 has been found to accelerate the development of CIA (collagen-induced arthritis) (Hom J T, et al., J. Immunol., 141: 834-41(1988)). IL-1 is present in the synovial fluid of patients with RA and in RA synovium (Nouri A M, et al., Clin. Exp. Immunol., 55: 295-302(1984); and MacNaul K L, et al., J. Immunol., 145: 4154-66(1990)).

The IL-1 receptor antagonist (IL-1Ra) is a natural protein that competitively inhibits the binding of IL-1β and IL-1α to IL-1 receptor types I and II in human and various animals, and improves the inflammatory symptoms of arthritis in experimental animal models (Kaklamanis P. M., Clin. Rheumatol., 11: 41-7(1992); Eisenberg S P, et al., Nature, 343: 341-6(1990); Smith R J, et al., Arthritis Rheum., 34: 78-83(1991); Hung G L, et al., Gene Ther., 1: 64-9(1994); Otani K, et al., J. Immunol., 156: 3558-62(1996)). Several independent clinical trials have been completed in which the recombinant IL-1Ra protein has been administered long term to patients with RA (Campion G V, et al., Arthritis Rheum., 39: 1092-101(1996); and Bresnihan B, et al., Arthritis Rheum., 41: 2196-204(1998)). The results indicate that treatment with IL-1Ra lowers both the levels of proteins involved in the acute-phase of RA and the counts of swollen joints, and furthermore, may inhibit radiographic progression of the disease (Bresnihan B. et al., Arthritis Rheum., 41: 2196-204(1998); and Jiang Y. et al., Arthritis Rheum., 43: 1001-9(2000)). However, daily repeated injections are needed to overcome the short half-life of this protein, and therefore, IL-1Ra as a protein form is impractical for clinical use (Wooley P H, et al., Arthritis Rheum., 36:1305-14(1993); Joosten L A, et al., Arthritis Rheum., 39: 797-809(1996); and Granowitz E V, et al., Cytokine, 4: 353-60(1992)). With the recent advances in gene therapy, the IL-1Ra gene has been delivered by retrovirus-based, adenovirus-based and adeno-associated virus (AAV)-based vectors into synoviocytes to achieve anti-inflammatory effects both in vivo and in vitro with a varying degree of success (Hung G L, et al., Gene Ther., 1: 64-9(1994); Otani K, et al., J. Immunol., 156: 3558-62(1996); Ghivizzani S C, et al., Proc. Natl. Acad. Sci., USA, 95: 4613-8(1998); Bakker A C, et al., Arthritis Rheum., 40: 893-900(1997); and Pan R Y, et al., Arthritis Rheum., 43: 289-97(2000)).

Among various viral and nonviral techniques for gene transfer in vivo, the direct injection of plasmid DNA into muscle is probably the simplest, most inexpensive and safest (Nishikawa M, et al., Hum. Gene Ther., 12: 861-70(2001)).

The present inventors have previously reported that gene transfer into leg muscles by directly injecting plasmid DNA in vivo can be used to deliver cytokines efficiently (Lee Y, et al., Biochem. Biophys. Res. Commun., 272: 230-5(2000)).

Therefore, the present inventors have further endeavored to develop a novel gene transfer delivery system of IL-1Ra for treating arthritis in a mammal, and, as a result, have discovered that muscular injection of an expression vector containing a DNA encoding the IL-1Ra is an excellent method for treating arthritis, therapeutic effect lasting for more than 14 days after a single treatment.

### SUMMARY OF THE INVENTION

Accordingly, it is an object of the present invention to provide a pharmaceutical composition for treating arthritis in a mammal.

It is another object of the present invention to provide a method for treating arthritis in a mammal.

In accordance with one aspect of the present invention, there is provided an intramuscular injection composition for treating arthritis in mammal, which comprises a DNA encoding interleukin-1 receptor antagonist and pharmaceutically acceptable carriers.

In accordance with another aspect of the present invention, there is provided a method for treating arthritis in a mammal, which comprises administering an effective amount of the DNA encoding interleukin-1 receptor antagonist thereto via intramuscular injection.

### BRIEF DESCRIPTION OF THE DRAWINGS

The above and other objects and features of the present invention will become apparent from the following description of the invention, when taken in conjunction with the accompanying drawings, in which:

FIG. 1 shows the levels of IL-1β in the knee joints of normal mice and mice having induced CIA;

FIG. 2 displays a structure of the inventive expression vector pCK-IL-1Ra (Hatched box: major immediate early promoter of human cytomegalovirus; dotted box: exon; wavy line: intron; pA: poly A tract; Kan: kanamycin resistance gene; and ColEI: E.coli origion of replication);

FIGS. 3A and 3B represent the time-dependent changes in the in vivo expression levels of human IL-1Ra in muscles (A) and serum (B), respectively, after intramuscular injection of pCK-IL-1Ra;

FIGS. 4A, 4B, 4C and 4D describe the effects of pCK-IL-1Ra on the CIA (A: Joint swelling, B: Arthritis, C: severe arthritis, and D: IL-β);

FIGS. 5A and 5B show how the level of pannus formation changes by the treatment of pCK-IL-1Ra;

FIG. 5C is a graph representing the results of FIGS. 5A and 5B;

FIGS. 6A and 6B show how the level of cartilage erosion changes by the treatment of pCK-IL-1Ra;

FIG. 6C is a graph representing the results of FIGS. 6A and 6B; and

FIG. 7 depicts the effect of administering the inventive composition on the incidence of arthritis.

### DETAILED DESCRIPTION OF THE INVENTION

In the inventive IL-1Ra (IL-1 receptor antagonist) gene therapy, the arthritis can effectively be treated by administering the DNA encoding IL-1Ra in the form of expression vector containing a DNA encoding IL-1Ra via intramuscular injection.

The DNA encoding IL-1Ra of the present invention can be obtained from human peripheral blood lymphocytes or synthesized using a conventional DNA synthesis method. Further, the DNA thus prepared may be inserted to a vector for intramuscular gene therapy, to obtain an expression vector.

The vector for intramuscular gene therapy that may be advantageously used in the present invention, is pCK plasmid which gives high level gene expression in the skeletal muscles of mice. pCK contains not only the full length major immediate-early(IE) promoter of human cytomegalovirus (HCMV) but also its entire 5′-untranslated region consisting of the entire exon 1 and intron 1, as well as a part of the exon 2. Further, pCK is designed in such a way that the start codon of the inserted gene coincides with the ATG codon of the original IE gene of HCMV, a feature unlike many other HCMV promoter-based expression vectors. The inventive expression vector which the human IL-1Ra is cloned to plasmid pCK is hereinafter refered to as “pCK-IL-1Ra”.

The present invention includes within its scope an intramuscular injection composition for treating arthritis comprising the DNA encoding IL-1Ra in the form of expression vector containing a DNA encoding IL-1Ra, preferably pCK-IL-1RA, in association with pharmaceutically acceptable carriers, excipients or other additives, if necessary.

Examples of suitable carriers, excipients, and diluents are lactose, dextrose, sucrose, sorbitol, mannitol, starches, gum acacia, alginates, gelatin, calcium phosphate, calcium silicate, cellulose, methyl cellulose, microcrystalline cellulose, polyvinylpyrrolidone, water, methylhydroxybenzoates, propylhydroxybenzoates, talc, magnesium stearate and mineral oil. The compositions may additionally include lubricating agents, wetting agents, flavoring agents, emulsifiers, preservatives and the like.

The compositions of the invention may be formulated so as to provide quick, sustained or delayed release of the active ingredient after their administration to a patient by employing any of the procedures well known in the art.

The present invention also includes within its scope a method for treating arthritis in a subject by way of administering a therapeutically effective amount of the DNA encoding IL-1Ra in the form of expression vector containing a DNA encoding IL-1Ra, preferably pCK-IL-1RA via intramuscular injection. The effective amount of the DNA encoding IL-1R as an active ingredient may range from about 0.05 to 500 mg/kg, preferably 0.5 to 50 mg/kg body weight, and can be administered in a single dose or in divided doses. However, it should be understood that the amount of the active ingredient actually administered ought to be determined in light of various relevant factors including the condition to be treated, the age and weight of the individual patient, and the severity of the patient's symptom; and, therefore, the above dose should not be intended to limit the scope of the invention in any way.

In the present invention, the intramuscular direct injection of the DNA encoding IL-1Ra works to generate a remarkable synergistic treatment effect than that achievable with any conventional administration of the compositions, which can be sustained for more than 14 days.

The following Examples are intended to further illustrate the present invention without limiting its scope.

Further, percentages given below for solid in solid mixture, liquid in liquid, and solid in liquid are on a wt/wt, vol/vol and wt/vol basis, respectively, and all the reactions were carried out at room temperature, unless specifically indicated otherwise.

### REFERENCE EXAMPLE 1

### Measurement of Levels of IL-1Ra and IL-1β

The levels of human IL-1Ra in the injected areas of muscles and serum and the levels of murine IL-1β in knees and ankles were measured using commercially available ELISA for human IL-1 Ra (R&D Systems, Minniapolis, Minn., USA) and mIL-1β (R & D Systems), according to the manufacturer's recommendations.

The injected areas of muscles were excised and homogenized in lysis buffer (25 mM Tris-HCl, pH 7.4, 50 mM NaCl, 0.5% Na-Deoxycholate, 2% NP-40, 0.2% SDS, 1 mM phenylmethylsulfonyl fluoride). While in the case of joint tissues, whole mice knees or ankles were snap frozen in liquid nitrogen and were ground into powder by pestle, then lysed with the lysis buffer. The supernatants containing total protein were used to measure the levels of cytokines. In the case of serum, it was directly subjected to IL-1Ra assays without any pre-treatment. The levels of IL-1Ra and IL-1β were normalized to the total amount of protein prepared from tissue lysates, as measured by way of a DC protein assay kit (Bio-Rad Laboratories, Hercules, Calif.).

### EXAMPLE 1

### Induction of Collagen-induced Arthritis (CIA)

20 nine-ten week-old DBA/1 mice (Charles River, Mass., USA) were intradermally immunized at the base of the tail with bovine type II collagen (100 μg; Chondrex, Wash.) emulsified in Freund's complete adjuvant (GIBCO BRL, NY). On day 21, the mice were boosted with an intradermal injection of 100 μg type II collagen. Gradual onset of arthritis normally starts approximately 4 weeks after initial immunization.

On day 40 after the initial immunization, the IL-1β levels in the knee joints were measured in accordance with the method of Reference Example 1 and compared with that of healthy mice.

FIG. 1 shows the level of IL-1β in the knee joints of mice having induced CIA. As shown in FIG. 1, a significant increase in the level of IL-1β was observed in knee joints of mice with CIA as compared with that of healthy mice (P<0.0001).

### EXAMPLE 2

### Cloning of Human IL-1Ra and Construction of Expression Vector

cDNA encoding human IL-1Ra was cloned from total RNA prepared from human peripheral blood lymphocytes by reverse transcription (RT)-polymerase chain reaction (PCR). PCR primers were:

(SEQ ID NO: 1)

5′-AAGCTTATGGAAATCTGCAGAGGCCTCCGCAGTCAC-3′
and

(SEQ ID NO: 2)

5′-GTCGACCTACTCGTCCTCCTGGAAGTAGAATTTGGT-3′.

The amplified cDNA was initially cloned into the PCR product cloning site of pGEM-72f(+) plasmid (Promega, Wis., USA). Following sequence confirmation, the human IL-1Ra cDNA was cloned into the HindIII site of the mammalian expression vector pCK (Lee Y, et al., Biochem. Biophys. Res. Commun., 272: 230-5(2000)) to obtain plasmid pCK-IL-1Ra, which was purified using an EndoFree plasmid Maxi prep kit (Qiagen,Valencia, Calif., USA), diluted in phosphate-buffered saline (pH 7.4) to 4 μg/μl and stored at −20° C. before use.

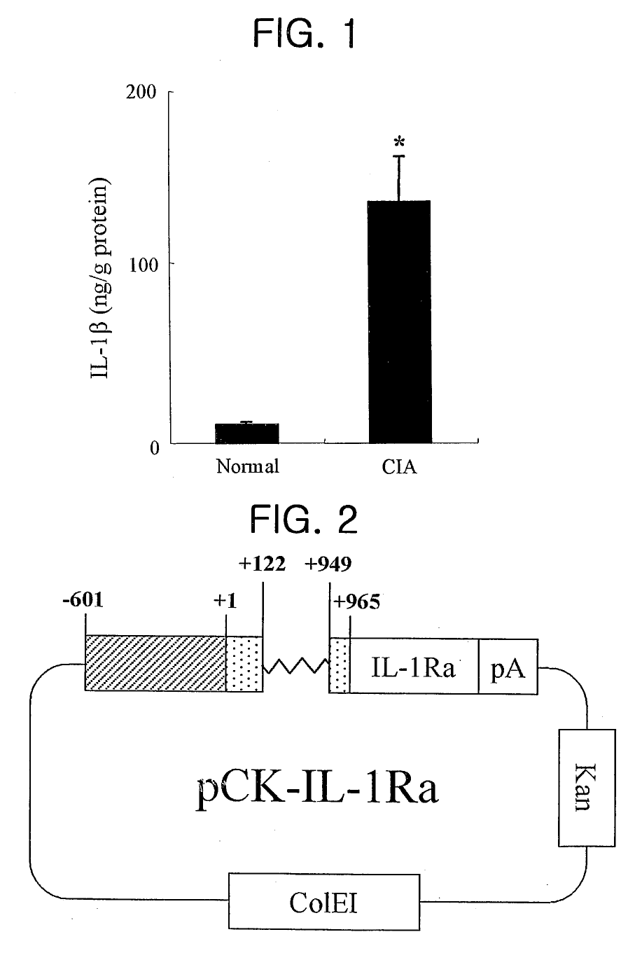

FIG. 2 displays the structure of the inventive expression vector pCK-IL-1Ra (Hatched box: major immediate early promoter of human cytomegalovirus; dotted box: exon; wavy line: intron; pA: poly A tract; Kan: kanamycin resistance gene; and ColEI: E.coli origion of replication).

### EXAMPLE 3

### Test of Time-dependent Changes In vivo Expression of IL-1Ra after Direct Injection of pCK-IL-1Ra

To investigate how long the expression of human IL-1Ra by delivered pCK-IL-1Ra can be sustained in the injected areas of muscles and serum, pCK-IL-1Ra prepared in Example 2 was injected into four sites (100 μg/25 μl at each of four sites, for a total of 400 μg/100 μl for each mouse) of the skeletal muscles of DBA/1 mice (Charles River, Mass., USA).

The levels of IL-1Ra produced in muscles and serum were determined for a period of 30 days using the method described in Reference Example 1.

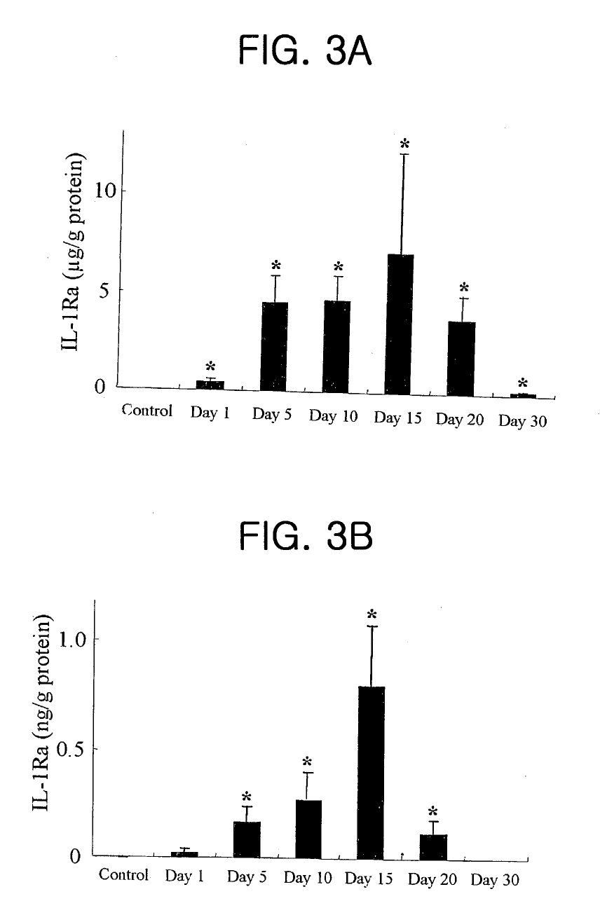

FIGS. 3A and 3B show the time-dependent changes in the in vivo expression levels of human IL-1Ra in muscles (A) and serum (B), respectively, after intramuscular injection of pCK-IL-1Ra.

As shown in FIG. 3A, the level of IL-1Ra in the injected areas was significantly high for 20 days. Further, as shown in FIG. 3B, significant levels of serum IL-1Ra were also detected for 20 days after injection of pCK-IL-1Ra, although the level of IL-1Ra in the serum was 1,000 times lower than in the injected muscle areas due to the dilution by circulating blood of IL-1Ra expressed in muscle cells.

### EXAMPLE 4

### Treatment Effect-Test of pCK-IL-1Ra on the CIA

CIA induction was carried out using 30 nine-ten week-old DBA/1 mice (Jackson Laboratory, Me., USA) in accordance with the method of Example 1.

Then, on day 30 after the initial immunization, mice that had not yet developed any macroscopic signs of arthritis, were chosen and divided into 2 groups. The mice of the two groups were each treated with 400 μg(100 μg/25 μl at each of four sites, for a total of 400 μg/100 μl for each mouse) of plasmid pCK-IL-1Ra prepared in Example 2 or a control plasmid, into four different sites (the thigh and calf muscles of two hind legs) using a 1-mL syringe equipped with a 27-gauge needle. A plasmid lacking the IL-1Ra coding sequence, pCK (Lee Y, et al., Biochem. Biophys. Res. Commun., 272: 230-5(2000) was used as the control plasmid.

On day 12 after the treatment of plasmid, macroscopic scoring of the paws and histologic analysis of the knees were evaluated. That is, erythema and swelling of the paws were scored on a 0-4 scale, with a maximum score of 4 for each paw. The >2 score was considered as incidence of arthritis. Two independent observers, without knowledge of the experimental groups performed scoring.

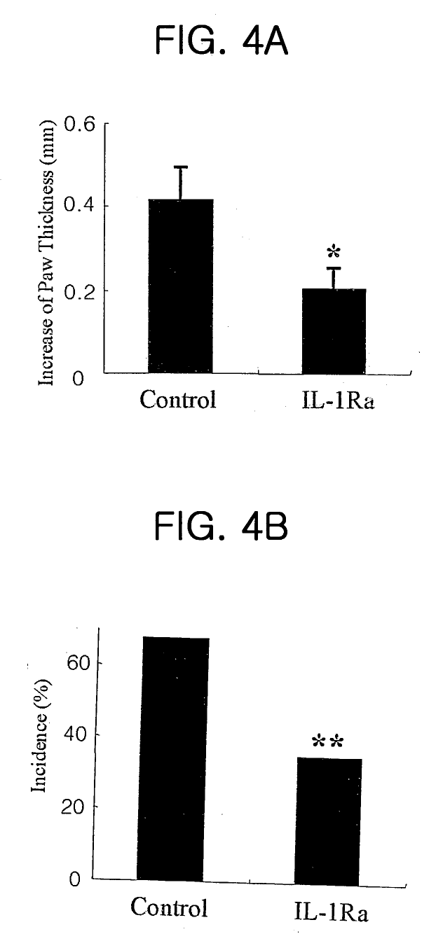

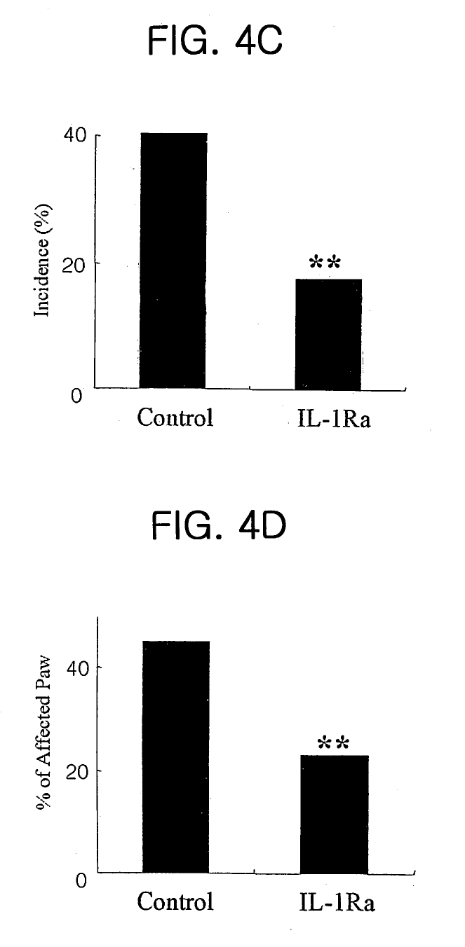

FIG. 4A, 4B, 4C and 4D describe effects of pCK-IL-1Ra on the CIA (A: Joint swelling, B: Arthritis, C: severe arthritis, and D: IL-β).

As can be seen from FIG. 4A, the increase of paw thickness was significantly smaller in mice treated with pCK-IL-1Ra, as compared with that of the control group. FIG. 4B shows that the incidence of arthritis was seen in 68% of the paws of the control group, while only 35% of the paws of those mice treated with pCK-IL-1Ra (P<0.05) showed the sign of arthritis. Similarly, in FIG. 4C, the incidence of severe arthritis (higher than index 3) was seen in 40% of the paws of the control group, versus only 18% of the paws of mice treated with pCK-IL-1Ra (P<0.05).

Further, the levels of proinflammatory cytokine, IL-1β in the paws were measured according to the method described in the Reference Example 1. Consistent with the joint swelling result, high levels of IL-1β (higher than 10 ng/gram of tissue protein) were found in 45% of the paws of the control group, versus 23% of the paws of mice treated with pCK-IL-1Ra (P<0.05) (FIG. 4D). These results suggest that intramuscular injection of pCK-IL-1Ra containing IL-1Ra gene can effectively suppress the incidence of murine collagen induced arthritis and relieve the severity of the disease.

For histologic analysis, knee joints were dissected, fixed in 10% phosphate-buffered formalin for 2 days, decalcified in 10% EDTA for 7 days, and then embedded in paraffin. Standard frontal sections of 7 μm were prepared, stained with either hematoxylin/eosin or Safranin O/fast green. Pannus formation was scored arbitrarily as 0 when no pannus formed in the joint space or 1-2 according to the degree of pannus formation. Cartilage depletion was visualized by diminished Safranin O-staining of the matrix and scored arbitrarily as 0 when normal, and 1-3 according to the degree of depletion (loss of staining).

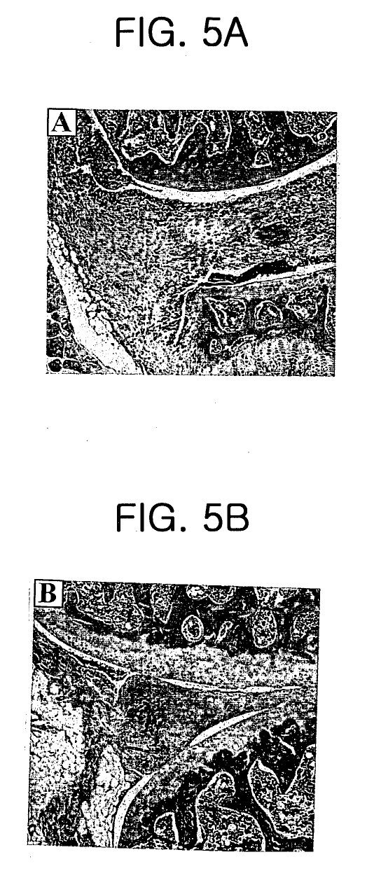

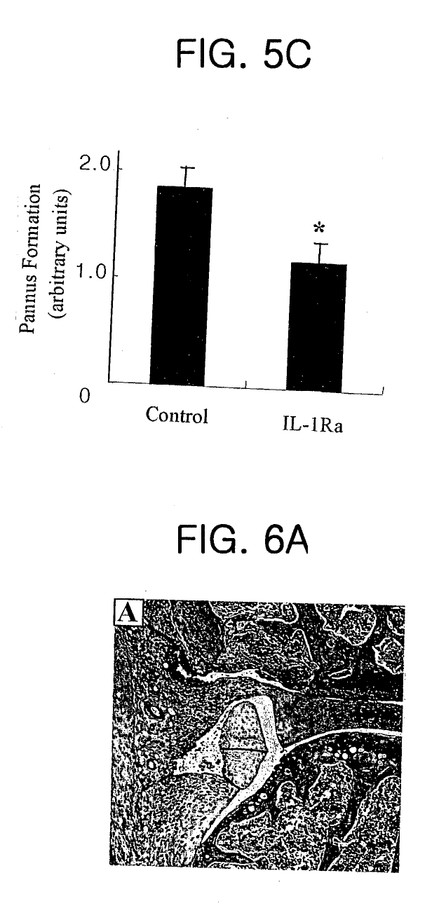

FIGS. 5A and 5B show how the pannus formation is affected by the treatment of pCK-IL-1Ra as compared with the control group. FIG. 5C is a graph representing shown the results of FIGS. 5A and 5B. The sections stained with hematoxylin and eosin in FIG. 5 show that the knee joints of mice treated with pCK-IL- 1Ra had significantly decreased pannus formation, as compared with the control.

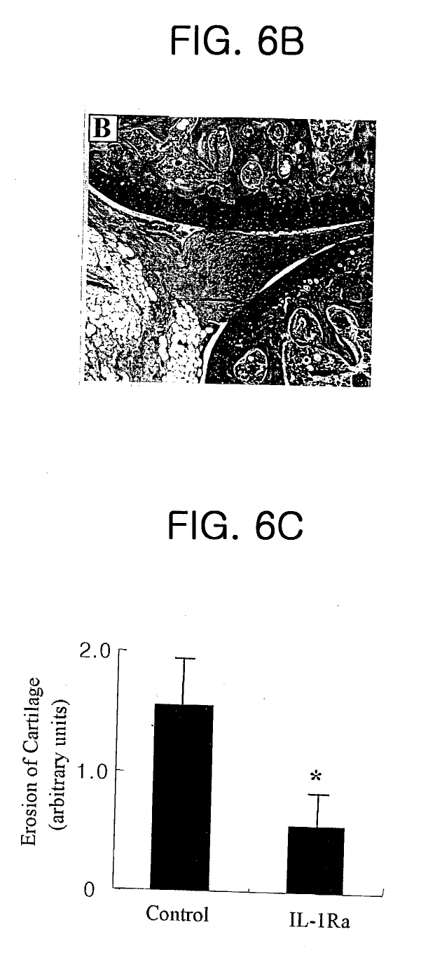

FIGS. 6A and 6B show how the cartilage erosion is suppressed by the treatment of pCK-IL-1Ra. FIG. 6C is a graph representing the results of FIGS. 6A and 6B.

As can be seen from FIG. 6, thinning and hyalinization of the cartilage were inhibited in the mice injected with pCK-IL-1Ra. Further, safranin O-staining of proteoglycan in the cartilage showed that the proteoglycan was well-preserved in the joints of mice treated with pCK-IL-1Ra, but not in those treated with control plasmid DNA. A statistically significant difference was found in the severity of cartilage erosion between the pCK-IL-1Ra-treated group and the control groups (P<0.05).

### EXAMPLE 5

### Test of Sustained Effects of Single Treatment of pCK-IL-1Ra

CIA induction was carried out using 30 nine-ten week-old DBA/1 mice (Jackson Laboratory, Me., USA) in accordance with the method of Example 1. On day 30 after the initial immunization, mice that had not yet developed any macroscopic signs of arthritis, were chosen and divided into 2 groups. The mice of the two groups were each treated with 400 μg of plasmid pCK-IL-1Ra prepared in Example 2 or a control plasmid, into the skeletal muscles. A plasmid lacking the IL-1Ra coding sequence, pCK, was used as the control plasmid.

The incidence of arthritis (>grade 2) in the paws was evaluated every three or four days until day 20 after treatment of plasmid.

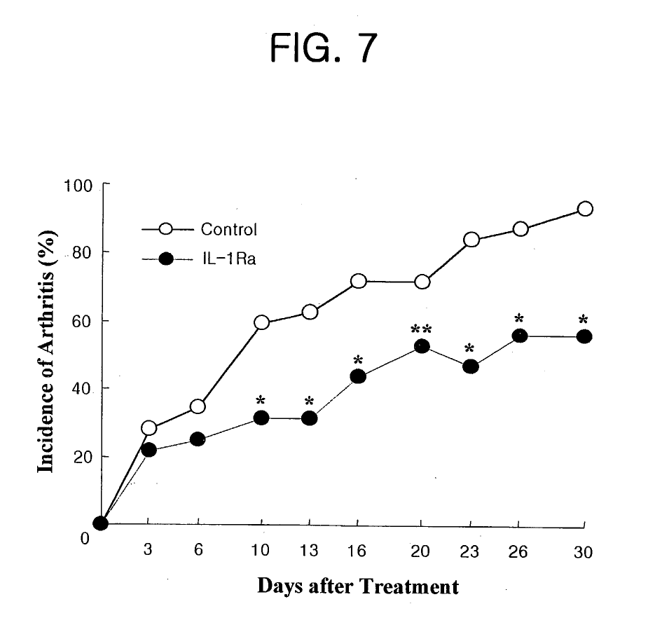

FIG. 7 depicts the time-dependent changes in the incidence of arthritis. As shown in FIG. 7, injection of pCK-IL-1Ra can efficiently prevent the occurrence of arthritis until 14 days after treatment (P<0.05). These results suggested that therapeutic effects of a single injection last for a minimum of 14 days after treatment, which is consistent with the result showing that a significant level of the hIL-1Ra gene expression was sustained for 15 days in the serum of mice treated with pCK-IL-1Ra in the Example 3.

While the invention has been described with respect to the above specific embodiments, it should be recognized that various modifications and changes may be made to the invention by those skilled in the art which also fall within the scope of the invention as defined by the appended claims.

What is claimed is:

1. An intramuscular injection composition for treating arthritis in a mammal, which comprises a DNA encoding interleukin-1 receptor antagonist (IL-1Ra) and pharmaceutically acceptable carriers.

2. The composition of claim 1, wherein the DNA encoding IL-1Ra is contained in an expression vector.

3. The composition of claim 2, wherein the expression vector is pCK-IL-1Ra.

4. The composition of claim 1, wherein the mammal is human.

5. A method for treating arthritis in a mammal, which comprises administering thereto an effective amount of a DNA encoding IL-1Ra via intramuscular injection.

6. The method of claim 5, wherein the DNA encoding IL-1Ra is contained in an expression vector.

7. The method of claim 6, wherein the expression vector is pCK-IL-1Ra.

8. The method of claim 5, wherein the mammal is human.

9. The method of claim 5, wherein the effective amount of the DNA encoding IR-1Ra ranges from 0.05 to 500 mg/kg body weight.
